(19) United States
(12) Patent Application Publication
(10) Pub. No.: US 2005/0130222 A1
(43) Pub. Date: Jun. 16, 2005

(54) Sample concentration maldi plates for maldi mass spectrometry

(76) Inventor: Peter Jeng Jong Lee, Westboro, MA (US)

Correspondence Address: EDWARDS & ANGELL, LLP, P.O. BOX 55874, BOSTON, MA, 02205, US

(21) Appl. No.: 10/479,296

(22) PCT Filed: May 24, 2002
(86) PCT No.: PCT/US02/16613

Related U.S. Application Data
(60) Provisional application No. 60/293,497, filed on May 25, 2001.

(51) Int. Cl. G01N 33/53; C12M 1/34; B01D 59/44

(52) U.S. Cl. 435/7.1; 435/287.1; 250/281

(57) ABSTRACT

A novel Matrix-Assisted Laser Desorption/Ionization Mass Spectrometry (MALDI-MS) sample support plate is described. The plate comprises a top sample presentation surface and a lower surface, wherein the top sample presentation surface comprises at least one aperture for receiving a sample. The aperture extends through and between the top sample presentation surface and the bottom surface, and contains a porous material that retains and concentrates at the target spot analyte and matrix molecules contained in the sample on the surface of the aperture. Methods for making and using the sample support plate in conventional and automated MALDI-MS are also described.

### BACKGROUND OF THE INVENTION

Mass spectrometry (MS) with ionization by matrix-assisted laser desorption and ionization (MALDI) has become a useful tool for the analysis of large molecules such as proteins, peptides, oligonucleotides, DNA, RNA, etc. It is well known that the sensitivity of the analysis and the speed of automation are highly dependent on preparation of the sample on the MALDI plate. The issues associated with sample preparation for MALDI Mass Spectrometry are summarized in published British patent application GB 2332273A.

For example, when a drop of sample and matrix solution is placed onto a clean metal sample support plate, the drop wets an area on the metal surface. After the solution dries, the sample spot consisting of small matrix crystals spreads over the formerly wet area. In general, the wetted area is not uniformly coated. In aqueous solutions, most of the small crystals of the matrix generally begin to grow at the margin of the wet area on the metal plate and continue to grow toward the center of the wet area. Thus, the analyte molecules are irregularly distributed, and the center of the spot is frequently devoid of crystals or covered with small, fine crystals that are practically useless for MALDI ionization because of the high concentration of alkali salts also present.

This type of coating requires visual observation of the sample using a microscope. Furthermore, the MALDI ionization yield and mass resolution fluctuate in the sample spot from site to site. In fact, it is often a troublesome process to find a favorable location on the sample spot with good analyte ion yield and good mass resolution. Consequently, high sample throughput automation of MALDI mass spectrometry analysis is hindered, if not impossible.

A number of devices have been developed in an attempt to alleviate the aforementioned difficulties. For example, published PCT application WO 96/40888, and related U.S. Pat. Nos. 6,004,770 and 6,093,770, disclose a sample presentation device having a surface-bound complex that includes at least one molecule that can chemically modify a biomolecule. A biomolecule is exposed to the surface-bound complex, and the chemically modified biomolecule resulting from such exposure is then analyzed by mass spectrometry, for example, MALDI-MS.

British patent application GB 2332273A describes a MALDI plate, coated with a Teflon-like hydrophobic coating having hydrophilic patches (“anchors”), that utilizes surface property (hydrophilic or hydrophobic) modification on the plate. After sample droplets are deposited onto the anchors, the droplets shrink during solvent evaporation, thereby centering themselves onto the anchor positions. Thus, MS detection sensitivity increases 10 to 100 times as compared to the conventional dried sample droplet preparation method described above, because the analyte is concentrated in smaller spots. The sample spots can be arranged in a precise grid to facilitate rapid, automated MALDI-MS. Such coated plates (Anchor Chip™) are marketed by Bruker Daltonics®.

Published PCT Application WO 01/19520 A1 describes a high density cast-in-place sample preparation card useful in a variety of analytical methods, including MALDI-MS. In particular, the patent application describes the use of the surface of a housing insert containing a plug of silica for direct MALDI-time of flight-MS analysis using a system designed by PE Biosystems. The system comprises a sample plate holder to hold the housing, and a Mass Spectrometer, designed to accommodate analysis using the sample plate holder.

However, many existing sample plates are limited to use with hydrophilic compounds or to specific MS instruments, require special surface modification, or have other disadvantages. Therefore, a need exists for a MALDI-MS sample plate that is capable of concentrating and locating a vast array of analytes in a small spot precisely segregating alkali salts and other undesired materials from the analyte and matrix, and being used in virtually any type of MALDI-MS instrument.

### SUMMARY OF THE INVENTION

The invention is directed to a sample support plate and method for concentrating a sample at one or more discrete locations for analysis by MALDI-MS. The invention also provides convenient methods for the preparation and use of the support plate. Additionally, the invention provides methods of sample preparation and analysis of the samples. Furthermore, the methods of sample preparation and analysis of the present invention are capable of concentrating and locating a large range of analytes in a small spot precisely, segregating salts and other undesired molecules from the analyte and matrix, and being useful for analysis in virtually any type of MALDI-MS.

Thus, in one aspect, the invention is a sample support plate for use in Matrix-Assisted Laser Desorption/Ionization Mass Spectrometry (MALDI-MS). The MALDI plate comprises a top sample presentation surface and a lower surface, wherein the top sample presentation surface comprises at least one aperture for receiving a sample. The aperture extends through and between the top sample presentation surface and the bottom surface, and contains a porous material that retains and concentrates analyte and matrix molecules contained in the sample on the surface of the aperture.

In a related aspect, the invention is a sample support plate for use in Matrix-Assisted Laser Desorption/Ionization Mass Spectrometry (MALDI-MS). The plate comprises a top sample presentation surface and a lower surface, wherein the top sample presentation surface comprises at least one aperture for receiving a sample. The aperture extends through and between the top sample presentation surface and the bottom surface. In addition, the aperture contains a porous monolith that retains and concentrates analyte and matrix molecules contained in the sample on the surface of the aperture.

In another related aspect, the invention is a monolithic sample support plate, for use in Matrix-Assisted Laser Desorption/Ionization Mass Spectrometry (MALDI-MS). The monolithic plate comprises a top sample presentation surface and a lower surface, wherein the top sample presentation surface comprises at least one aperture for receiving a sample. The aperture extends through and between the top sample presentation surface and the bottom surface. In addition, the aperture contains a porous monolith that retains and concentrates analyte and matrix molecules contained in the sample on the surface of the aperture.

In another aspect, the invention is a method for preparing a sample for Matrix-Assisted Laser Desorption/Ionization Mass Spectrometry (MALDI-MS). The method comprises: providing the sample support plate described above; applying a sample to the aperture; and allowing selected molecules in the sample to penetrate or pass through the porous material in the aperture, and allowing other selected molecules in the sample to be retained on top of the porous material, thereby concentrating the other selected molecules on the surface of the aperture.

In yet another aspect, the invention is a method for preparing the sample support plate of the invention described above. The method comprises: providing a sample support plate comprising a top sample presentation surface and a lower surface; forming on the top sample presentation surface at least one aperture for receiving a sample, wherein the aperture extends through and between the top sample presentation surface and the bottom surface; and applying a porous material to the aperture.

In a related aspect, the invention is directed to a method for preparing the sample support plate of the invention by: forming, on a sample support plate having a top sample presentation surface and a bottom surface, at least one aperture on the sample presentation surface, such that the aperture extends through and between the top sample presentation surface and bottom surface; and applying a porous material to the aperture.

Another aspect of the invention is a method for performing Matrix-Assisted Laser Desorption/Ionization Mass Spectrometry (MALDI-MS) on an analyte of interest. The method comprises: providing a sample support plate, wherein the plate comprises a top sample presentation surface and a lower surface, wherein the top sample presentation surface comprises at least one aperture for receiving a sample, and wherein the aperture extends through and between the top sample presentation surface and the bottom surface, and contains a porous material that retains and concentrates analyte and matrix molecules contained in the sample; applying a sample comprising an analyte of interest to the aperture; and allowing selected molecules in the sample to penetrate or pass through the porous material in the aperture, and allowing the analyte of interest in the sample to be retained on top of the porous material, thereby concentrating the analyte of interest on the surface of the aperture; and performing MALDI-MS on the analyte of interest.

### BRIEF DESCRIPTION OF THE DRAWINGS

FIG. 1A shows side and top views of a sample support plate with an aperture.

FIG. 1B shows side and top view of a sample support plate with an aperture containing a porous monolith.

FIG. 1C shows a sample support plate, with an aperture containing a porous monolith, and a sample applied to the aperture, positioned on a vacuum manifold.

FIG. 2A shows a top view of a portion of a sample support plate with three sample target spots and associated apertures containing porous sorbent.

FIG. 2B shows a side view of the sample support plate of FIG. 2A.

### DETAILED DESCRIPTION OF THE INVENTION

The invention is directed to a flow-through, sample-concentrating support plate for use in MALDI-MS. The invention is also directed to convenient methods of preparation and use of the support plate, as well as to methods of sample preparation and analysis of the samples. The sample support plate of the invention, and associated methods, provide a number of advantages as follows.

The analyte and matrix molecules can be concentrated and precisely located in a small area.

The porous material contained in the aperture can capture and concentrate the analyte molecules, and therefore large volumes of highly dilute sample solution can be applied to the aperture to accumulate sufficient concentration of analyte molecules on the top of the porous material for effective and reliable mass spectrometry analysis.

When a biological solution passes through the porous material, the analyte molecules contained in the solution, for example, biomolecules, such as proteins, peptides, polynucleotides, etc., will be retained and concentrated on the top of porous material, and salts typically associated with aqueous solutions of such biomolecules, penetrate and/or pass through the porous material. Because the laser used in a MALDI mass spectrometer is applied only to the top of the aperture, or an aperture contained in a target spot, the salts will not interfere with the MALDI-MS ionization.

The invention also provides a MALDI-MS sample plate containing a plurality of target spots and associated apertures containing a porous material in a precise, grid format. The grid will enable high throughput automated MALDI-MS analysis.

These and other advantages and features of the invention are described in further detail below.

Definitions

Before further description of the invention, certain terms employed in the specification, examples, and appended claims are, for convenience, collected here.

The terms “sample support plate” or “sample plate” as used herein refer to an apparatus containing the target spots, on which a sample is placed for analysis by MALDI-MS. In certain embodiments, the sample support plate is monolithic; i.e., the plate is of unitary construction of a single material, e.g., a metal, a plastic, or a polymer. In certain other embodiments, the plate is metal, e.g. stainless steel. However, the sample support plate of the invention is not a housing, an insert, or a unit comprising a housing and an insert bonded together, as described in published PCT application WO 01/19520. In a preferred embodiment, the sample support plate is a conventional, stainless steel sample support plate that is provided with one or more apertures in one or more target spots, respectively.

The term “target spot” as used herein refers to the designated area of the sample support plate for the analysis of one particular sample or a mixture of samples.

The term “aperture” as used herein refers to a hole in the support plate, e.g., a hole in a target spot, in which a porous material is added. The size of the aperture at the sample presentation surface defines the size of the analysis surface or zone for MALDI-MS analysis. The size and shape of the aperture may be used to control the extent of concentration of the analyte of interest and the matrix by modifying the size of the aperture; e.g., an increase in the size of the aperture decreases the extent to which concentration can occur by increasing the analysis surface area of the porous material contained within the aperture, and vice versa.

The terms “porous material” or “sufficiently porous” as used herein refer to any material, prepared or inserted within the aperture, that is capable of allowing selected molecules, e.g., salts, solvents and combinations thereof, to penetrate and/or pass through the material, and preventing other selected materials, e.g., an analyte of interest and/or a matrix material, from penetrating the material, such that the other selected materials are retained on the surface of the porous material. In this manner, the porous material concentrates the analyte of interest on the surface of the aperture. Thus, the term “porous material” is intended to include porous sorbents, membranes, filters, and other filtering means. In certain embodiments, the porous sorbent comprises a bed of particles or a porous monolith. The term “porous monolith” as used herein refers to a continuous plug of chromatographic material as distinguished from a bed of individual particles. Examples of porous monoliths include macroporous polymer plugs as described in U.S. Pat. No. 5,334,310. Furthermore, the term “porous monolith” and “bed of particles” are distinguished from the terms “membrane” and “composite structure” as defined in WO 01/19520 A1.

The term “salts” as used herein refers to any molecule, including alkali salts, that adversely affects the quality of the mass spectrum of an analyte of interest because of adduct formation.

The term “penetration” as used herein refers to the movement of selected molecules into or through the porous material by absorption, adsorption and/or simple filtration. The term is contrasted with “retention” which refers to selected molecules that do not do not penetrate the porous material and therefore remain or are “retained” on the surface of the porous material.

The term “analyte of interest” as used herein refers to the molecule or molecules that are to be analyzed, for example, by MALDI-MS.

The term “matrix material” as used herein refers to any material suitable for use in MALDI-MS, and includes one or more small, acidic, light absorbing chemicals, e.g., nicotinic or sinapinic acid, that are mixed in solution with the analyte in such a manner so that upon drying on the target spot, the crystalline matrix-embedded analyte molecules are successfully desorbed (by laser irradiation) and ionized from the solid phase crystals into the gaseous or vapor phase and accelerated as molecular ions. A large fold excess of the matrix material facilitates crystal formation and entrapment of analyte.

### DESCPRIPTION OF CERTAIN EMBODIMENTS OF THE INVENTION

In one aspect, the invention is directed to a sample support plate for use in Matrix-Assisted Laser Desorption/Ionization Mass Spectrometry (MALDI-MS). The MALDI plate comprises a top sample presentation surface and a lower surface, wherein the top sample presentation surface comprises at least one aperture for receiving a sample. The aperture extends through and between the top sample presentation surface and the bottom surface, and contains a porous material that retains and concentrates analyte and matrix molecules contained in the sample on the surface of the aperture. In another aspect, the invention is directed to the methods of sample support plate preparation, sample preparation, and analysis.

The aperture can be of any size and shape. The size of the aperture at the sample presentation surface defines the size of the analysis surface for MALDI-MS analysis. Further, the size and shape of the aperture may be used to control the extent of concentration by modifying the size of the aperture, e.g., an increase in the size of the aperture decreases the extent to which concentration can occur by increasing the analysis surface area of the porous material contained within the aperture. In one embodiment, the aperture is oriented through the plate in a vertical path, perpendicular to the surface of the plate. In certain embodiments the aperture is substantially cylindrical in shape and in other embodiments, the aperture is substantially conical in shape. In preferred embodiments, the aperture is located within a target spot on the sample presentation surface of the plate.

The porous material is sufficiently porous to allow penetration of selected materials in or through the porous material and/or retention on the porous material of other selected molecules. In specific embodiments, the selected molecules that penetrate the porous material include salts, solvents and combinations thereof, and the other selected molecules that are retained on the surface of the porous material include the analyte of interest and the matrix.

In certain embodiments, the porous material is selected from a porous monolith and a bed of particles. In a particular embodiment, the porous material is a porous monolith. In a preferred embodiment, the porous monolith is a macroporous polymeric plug.

A porous monolith in accordance with the invention can be prepared by admixing a monomer, a porogen, and an initiator. Specific examples of the monomer include monovinyl monomers, polyvinyl monomers, or a mixture of monovinyl and polyvinyl monomers. Monovinyl monomers include, for example, styrene, N-vinylpyrrolidone, methacrylate, vinylacetate, glycidyl methacrylate, or any combination thereof. Polyvinyl monomers include, for example, divinylbenzene, ethylene dimethacrylate, bis-acrylamide, divinylpyridine, ethylene dimethacrylate, hydroxyalkylene dimethacrylate, or any combination thereof. Exemplary porogens include aliphatic hydrocarbons, aromatic hydrocarbons, esters, alcohols, ketones, ether, or any combination thereof. Examples of initiators include benzoyl peroxide, lauroyl peroxide, peroxodisulfate, Vazo 52, Vazo 64, Vazo 67, Vazo 88, V70, or any combination thereof.

In specific embodiments, the aperture is in a precisely defined location. The precisely defined location of the aperture facilitates automated analysis. In certain embodiments of the invention, the sample plate comprises a plurality of apertures, such that a grid of apertures is formed on the top sample presentation surface. In even more specific embodiments, the grid comprises 96 apertures. Moreover, the plurality of apertures facilitates high throughput analysis.

In one embodiment of the invention, a vacuum is applied to assist in the penetration of selected molecules into or through the porous material in the aperture. In certain embodiments, the vacuum is applied by placing the sample support plate on a vacuum manifold.

Another aspect of the invention is a method for preparing a sample for Matrix-Assisted Laser Desorption/Ionization Mass Spectrometry (MALDI-MS). Using a sample support plate described above, a sample is applied to the aperture and selected molecules in the sample are allowed to penetrate or pass through the porous material in the aperture. Other selected molecules in the sample are retained on top of the porous material, thereby concentrating the other selected molecules on the surface of the aperture. In certain embodiments, a vacuum is applied to the sample support plate prior to applying the sample to the aperture. In particular embodiments, the vacuum is applied by placing the sample support plate on a vacuum manifold.

In one embodiment of the method, the sample comprises an analyte of interest, a matrix material, one or more salts, and one or more solvents. The sample is applied to the aperture, and then a vacuum is applied at a rate that allows the sample to pass into or through the sorbent material gradually, thus allowing and/or causing one or more of the salts and one or more the solvents to pass into or through the porous material, and allowing and/or causing the analyte of interest and the matrix material to be retained on top of the porous material, thereby concentrating the analyte of interest on the surface of the aperture.

In another embodiment, the sample is a solution of a matrix material. The solution of matrix material is applied to the aperture, and then a vacuum is applied at a rate that allows the sample to pass into or through the sorbent material gradually, thus allowing and/or causing the matrix material to be retained on top of the porous material and form crystals on the top of the porous material, thereby concentrating crystals of the matrix material on the surface of the aperture. A second sample containing a solution of an analyte of interest is applied to the aperture, and then a vacuum is applied at a rate that allows and/or causes the second sample to pass into or through the sorbent material gradually, thus allowing and/or causing the analyte of interest to be retained on top of the porous material, thereby concentrating the analyte of interest on top of the porous material and incorporating the analyte of interest with the crystals of matrix material already retained on top of the porous material on the surface of the aperture.

In yet another embodiment, the sample is a solution of the analyte of interest. The solution of the analyte of interest is applied to the aperture, and then a vacuum is applied at a rate that allows and/or causes the sample to pass into or through the porous material gradually, thus allowing and/or causing the analyte of interest to be retained on top of the porous material, thereby concentrating the analyte of interest on the surface of the aperture. A second sample containing a solution of a matrix material is then applied to the aperture, and a vacuum is applied at a rate that allows and/or causes the second sample of the matrix material to pass into or through the porous material gradually, thus allowing and/or causing the matrix material to be retained on top of the porous material and form crystals on the top of the porous material, thereby concentrating crystals of the matrix material and incorporating the matrix material with the analyte of interest already retained on top of the porous material on the surface of the aperture. In an advantageous embodiment, water is applied to the aperture prior to applying the second sample containing the solution of the matrix material, and vacuum is applied to allow the water to pass through the porous material. Upon drying of the aperture, the plate is inserted into a MALDI mass spectrometer wherein the crystalline matrix-embedded analyte molecules are successfully desorbed (by laser irradiation) and ionized from the solid phase crystals into the gaseous or vapor phase and accelerated as molecular ions.

In another aspect, the invention is a method for preparing the sample support plate as described above. Just about any material can be used to make the sample support plate, but the material used should not deleteriously react with the reagents used for preparation of the sample, and also should be able to withstand the conditions typically used during MALDI-MS. Suitable materials include plastics (for example, polyolefins, especially polyethylene and polypropylene, PVC and polystyrene), glass and metal (for example, stainless steel). In preferred embodiments, the sample plate is monolithic. In other preferred embodiments, the sample support plate is a conventional sample plate that is typically used for MALDI-MS, and therefore is not limited by specific instrumentation design. At least one sample target spot is formed on the plate by creating an aperture, for example by drilling, for receiving the sample. The aperture extends through and between the top sample presentation surface and the bottom surface.

A plurality of apertures can be created in a grid pattern to facilitate automated, high throughput sample preparation and analysis. A 96 aperture plate, for example twelve parallel rows of eight apertures, is particularly advantageous for this purpose. In a preferred embodiment, the aperture is located in a target spot on the sample presentation surface of the plate.

A porous material is then applied to the aperture. In certain embodiments, the aperture is substantially completely filled with the porous material. In other embodiments, the aperture is partially filled with the porous material. One of skill in the art will readily appreciate that the type of porous material to be used will depend on the type of analyte, and therefore many types of porous materials, including a porous monolith or a bed of particles, can be used in accordance with the invention. one embodiment, the porous material is a porous monolith, as described above. In a preferred embodiment, the porous monolith is a macroporous polymer plug.

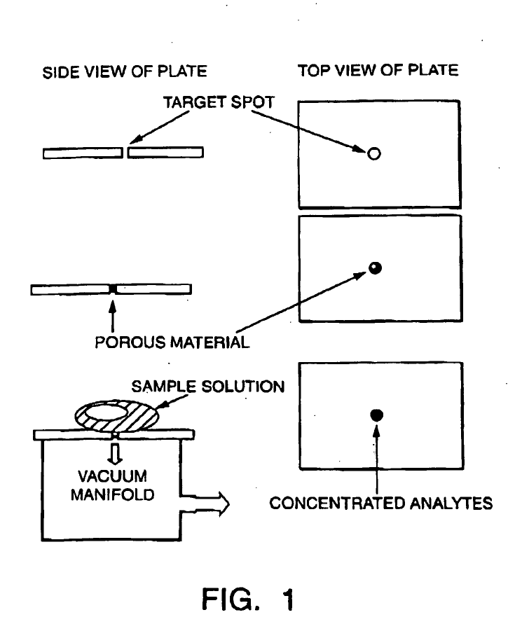

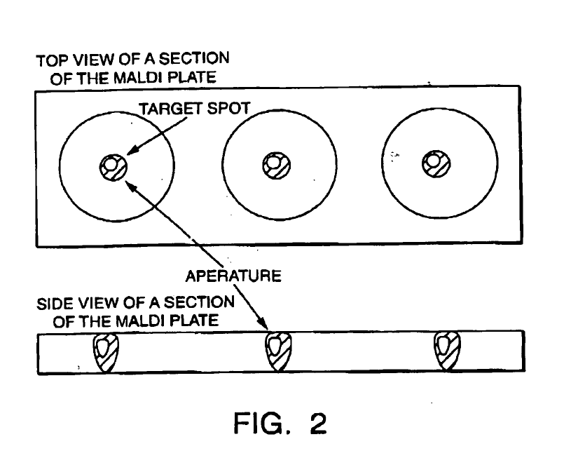

Particularly advantageous embodiments of the sample support plates of the invention are depicted in FIGS. 1 and 2 which show a monolithic, three dimensional plate design.

### EXEMPLIFICATION OF THE INVENTION

The invention is further illustrated by the following examples that should not be construed as limiting.

### Example 1

### Preparation of a Sample Support Plate of the Invention

A sample support plate, as shown in FIG. 2, is prepared as follows. A plurality of holes are drilled in a conventional steel MALDI plate having an equal number of target spots. The holes are then filled with a polymeric porous sorbent. The polymeric porous sorbent is prepared by admixing a solution containing a monomer, a porogen, and an initiator. The polymerization reaction is then initiated to create a porous polymer sorbent plug. The plate is placed on a vacuum manifold, and a vacuum is applied to the MALDI plate using a vacuum manifold. The porous polymer sorbent plug is then washed with appropriate solvents to remove the residues of monomer, porogen, and initiator.

### Example 2

### Sample Preparation Methods of the Invention

Method One

a) A sample support plate for use in MALDI-MS, prepared as described in Example 1, is placed on a vacuum manifold.

b) The vacuum is applied.

c) A sample solution containing the analyte of interest and the matrix is added on the top of the porous polymeric sorbent plug contained in each of the apertures in the target spots.

d) The sample solution is allowed to pass through the porous polymeric sorbent plug gradually, and at the same time the analyte molecules and matrix molecules are concentrated and incorporated on the top of porous polymeric sorbent plug.

e) Following drying, the plate is ready for insertion into the MALDI mass spectrometer.

Method Two

a) A sample support plate for use in MALDI-MS, prepared as described in Example 1, is placed on a vacuum manifold.

b) The vacuum is applied.

c) A matrix solution is added on the top of the porous polymeric sorbent plug contained in each of the apertures in the target spots.

d) The solution is allowed to pass through the porous polymeric sorbent plug gradually, and at the same time the matrix molecules are retained on the top of the porous polymeric sorbent plug and concentrated, to form small crystals on the top of the porous polymeric sorbent plug.

e) A solution containing the analyte of interest is added on the top of the porous polymeric sorbent plug.

f) The solution is allowed to pass through the porous polymeric sorbent plug gradually and at the same time the analyte molecules are concentrated on the top of porous material and are incorporated with crystalline matrix molecules.

g) Following drying, the plate is ready for insertion into the MALDI mass spectrometer.

Method Three

a) A sample support plate for use in MALDI-MS, prepared as described in Example 1, is placed on a vacuum manifold.

b) The vacuum is applied.

c) A solution containing the analyte of interest is added on the top of the porous polymeric sorbent plug contained in each of the apertures in the target spots.

d) The solution is allowed to pass through the porous polymeric sorbent plug gradually, and at the same time the analyte molecules are retained on the top of the porous polymeric sorbent plug and concentrated.

e) A solution containing a matrix is added on the top of the porous polymeric sorbent material.

f) The solution is allowed to pass through the porous material gradually and at the same time the matrix molecules are retained and concentrated on the top of porous polymeric sorbent plug, and form small crystals that incorporate the analyte molecules.

g) Following drying, the plate is ready for insertion into the MALDI mass spectrometer.

Method Four

Same as Method 3, except that prior to adding the matrix solution, water is added to the top of the porous polymeric sorbent material and allowed to pass through the material

### Incorporation by Reference

The entire contents of all patents, published patent applications and other references cited herein are hereby expressly incorporated herein in their entireties by reference.

### Equivalents

Those skilled in the art will recognize, or be able to ascertain, using no more than routine experimentation, many equivalents to specific embodiments of the invention described specifically herein. Such equivalents are intended to be encompassed in the scope of the following claims.

1. A sample support plate, for use in Matrix-Assisted Laser Desorption/Ionization Mass Spectrometry (MALDI-MS), comprising a top sample presentation surface and a lower surface, wherein the top sample presentation surface comprises at least one aperture for receiving a sample, wherein the aperture extends through and between the top sample presentation surface and the bottom surface, and wherein the aperture contains a porous material that retains and concentrates analyte and matrix molecules contained in the sample on the surface of the aperture.

2. The sample plate of claim 1, wherein the porous material is sufficiently porous to allow penetration in or through the porous material or retention on the porous material of selected molecules.

3. The sample plate of claim 2, wherein the porous material is selected from the group consisting of a porous monolith and a bed of particles.

4. The sample plate of claim 2, wherein the molecules that penetrate the porous material are selected from the group consisting of salts, solvents and combinations thereof, and the molecules that are retained on the surface of the porous material are the analyte and the matrix.

5. The sample plate of claim 3, wherein the porous material is a porous monolith.

6. The sample plate of claim 5, wherein the porous monolith is prepared by admixing, a monomer, a porogen, and an initiator.

7. The sample plate of claim 6, wherein the monomer is selected from the group consisting of a monovinyl monomer, a polyvinyl monomer, and a mixture of monovinyl and polyvinyl monomers.

8. The sample plate of claim 7, wherein the monovinyl monomer is selected from the group consisting of styrene, N-vinylpyrrolidone, methacrylate, vinylacetate, glycidyl methacrylate, and any combination thereof.

9. The sample plate of claim 7, wherein the polyvinyl monomer is selected from the group consisting of divinylbenzene, ethylene dimethacrylate, bis-acrylamide, divinylpyridine, ethylene dimethacrylate, hydroxyalkylene dimethacrylate, and any combination thereof.

10. The sample plate of claim 6, wherein the porogen is selected from the group consisting of aliphatic hydrocarbons, aromatic hydrocarbons, esters, alcohols, ketones, ether, and any combination thereof.

11. The sample plate of claim 6, wherein the initiator is selected from the group consisting of benzoyl peroxide, lauroyl peroxide, peroxodisulfate, Vazo 52, Vazo 64, Vazo 67, Vazo 88, V70, and any combination thereof.

12. The sample plate of claim 1, wherein the aperture is in a precisely defined location.

13. The sample plate of claim 7, wherein the precisely defined location of the aperture facilitates automated analysis.

14. The sample plate of claim 12, comprising a plurality of apertures, such that a grid of target spots is formed on the top sample presentation surface.

15. The sample plate of claim 14, wherein the grid comprises 96 apertures.

16. The sample plate of claim 14, wherein the plurality of apertures facilitates high through-put analysis.

17. The sample plate of claim 1, wherein the aperture is oriented through the plate in a vertical path, perpendicular to the surface of the plate.

18. The sample plate of claim 2, wherein a vacuum is applied to the aperture to assist in the penetration of selected molecules into or through the porous material.

19. The sample plate of claim 18, wherein the vacuum is applied using a vacuum manifold.

20. A method for preparing a sample for Matrix-Assisted Laser Desorption/Ionization Mass Spectrometry (MALDI-MS) comprising:
providing the sample support plate of claim 1;
applying a sample to the aperture; and
allowing selected molecules in the sample to penetrate or pass through the porous material in the aperture, and allowing other selected molecules in the sample to be retained on top of the porous material, thereby concentrating the other selected molecules on the surface of the aperture;
to thereby prepare a sample for MALDI-MS.

21. The method of claim 20, further comprising applying a vacuum to the sample support plate prior to applying the sample to the aperture.

22. The method of claim 21, wherein the vacuum is applied by placing the sample support plate on a vacuum manifold.

23. The method of claim 21, wherein the sample comprises an analyte of interest, a matrix material, one or more salts and one or more solvents.

24. The method of claim 23, wherein vacuum is applied at a rate that allows the sample to pass into or through the sorbent material gradually, thereby allowing one or more salts and one or more solvents to pass into or through the porous material and allowing the analyte of interest and the matrix material to be retained on top of the porous material, thereby concentrating the analyte of interest on the surface of the aperture.

25. The method of claim 21, wherein the sample contains a solution of a matrix material.

26. The method of claim 25, wherein vacuum is applied at a rate that allows the sample to pass into or through the porous material gradually, thereby allowing the matrix material to be retained on top of the porous material and form crystals on the top of the porous material, thereby concentrating crystals of the matrix material on the surface of the aperture.

27. The method of claim 26, further comprising applying a second sample containing a solution of an analyte of interest to the aperture, and applying a vacuum at a rate that allows the second sample to pass into or through the porous material gradually, thereby allowing the analyte of interest to be retained on top of the porous material, thereby concentrating the analyte of interest on top of the porous material and incorporating the analyte of interest with the crystals of matrix material on the surface of the aperture.

28. The method of claim 21, wherein the sample contains a solution of an analyte of interest.

29. The method of claim 28, wherein vacuum is applied at a rate that allows the sample to pass into or through the porous material gradually, thereby allowing the analyte of interest to be retained on top of the porous material, thereby concentrating the analyte of interest on the surface of the aperture.

30. The method of claim 29, further comprising applying a second sample containing a solution of a matrix material to the aperture, and applying a vacuum at a rate that allows the second sample to pass into or through the porous material gradually, thereby allowing the matrix material to be retained on top of the porous material and form crystals on the top of the porous material, thereby concentrating crystals of the matrix material and incorporating the matrix material with the analyte of interest on the surface of the aperture.

31. The method of claim 30, wherein prior to applying the second sample containing the solution of the matrix material, water is applied to the aperture, and vacuum is applied to allow the water to pass through the porous material.

32. A method for preparing the sample support plate of claim 1 comprising:
providing a sample support plate comprising a top sample presentation surface and a lower surface;
forming on the top sample presentation surface at least one aperture for receiving a sample, wherein the aperture extends through and between the top sample presentation surface and the bottom surface; and
applying a porous material to the aperture.

33. A method for preparing the sample support plate of claim 1 comprising:
forming, on a sample support plate having a top sample presentation surface and a bottom surface, at least one aperture, such that the aperture extends through and between the top sample presentation surface and bottom surface; and
applying a porous material to the aperture.

34. The method of claim 32, wherein the porous material comprises a porous monolith.

35. The method of claim 34, further comprising
admixing a monomer, a porogen and an initiator;
filling the aperture with the admixture;
initiating a polymerization reaction to form a porous monolith plug; and
washing the porous polymer sorbent plug to remove residual monomer, porogen and initiator.

36. The method claim 35, wherein the monomer is selected from the group consisting of a monovinyl monomer, a polyvinyl monomer, and a mixture of monovinyl and polyvinyl monomers.

37. The method of claim 36, wherein the monovinyl monomer is selected from the group consisting of styrene, N-vinylpyrrolidone, methacrylate, vinylacetate, glycidyl methacrylate, and any combination thereof.

38. The method of claim 36, wherein the polyvinyl monomer is selected from the group consisting of divinylbenzene, ethylene dimethacrylate, bis-acrylamide, divinylpyridine, ethylene dimethacrylate, hydroxyalkylene dimethacrylate, and any combination thereof.

39. The method of claim 35, wherein the porogen is selected from the group consisting of aliphatic hydrocarbons, aromatic hydrocarbons, esters, alcohols, ketones, ether, and any combination thereof.

40. The method of claim 39, wherein the initiator is selected from the group consisting of benzoyl peroxide, lauroyl peroxide, peroxodisulfate, Vazo 52, Vazo 64, Vazo 67, Vazo 88, V70, and any combination thereof.

41. A sample plate of claim 1, wherein the aperture may be any shape that allows the penetration of selected molecules and the retention of other selected molecules.

42. A method for performing Matrix-Assisted Laser Desorption/Ionization Mass Spectrometry (MALDI-MS) on an analyte of interest, comprising:
providing a sample support plate, wherein the plate comprises a top sample presentation surface and a lower surface, wherein the top sample presentation surface comprises at least one aperture for receiving a sample, and wherein the aperture extends through and between the top sample presentation surface and the bottom surface, and contains a porous material that retains and concentrates analyte and matrix molecules contained in the sample on the surface of the aperture;
applying a sample comprising an analyte of interest to the aperture;
allowing selected molecules in the sample to penetrate or pass through the porous material in the aperture, and allowing the analyte of interest in the sample to be retained on top of the porous material, thereby concentrating the analyte of interest on the surface of the aperture; and
performing MALDI-MS on the analyte of interest.

43. A sample support plate, for use in Matrix-Assisted Laser Desorption/Ionization Mass Spectrometry (MALDI-MS), comprising a top sample presentation surface and a lower surface, wherein the top sample presentation surface comprises at least one aperture for receiving a sample, wherein the aperture extends through and between the top sample presentation surface and the bottom surface, and wherein the aperture contains a porous monolith that retains and concentrates analyte and matrix molecules contained in the sample on the surface of the aperture.

44. A sample plate of claim 5, wherein the porous monolith is a macroporous polymer plug.

45. The method or sample plate of claim 1, wherein the top sample presentation surface comprises at least one sample target spot wherein the aperture is located within the target spot.

46. A sample support plate, for use in Matrix-Assisted Laser Desorption/Ionization Mass Spectrometry (MALDI-MS), comprising a monolithic support plate having a top sample presentation surface and a lower surface, wherein the top sample presentation surface comprises at least one aperture for receiving a sample, wherein the aperture extends through and between the top sample presentation surface and the bottom surface, and wherein the aperture contains a porous monolith that retains and concentrates analyte and matrix molecules contained in the sample on the surface of the aperture.
